(19) United States
(12) Patent Application Publication
(10) Pub. No.: US 2025/0361366 A1
(43) Pub. Date: Nov. 27, 2025

(54) INJECTABLE RADIOPAQUE CROSSLINKED HYDROGEL PARTICLE SUSPENSIONS AND METHODS OF FORMING SAME

(71) Applicant: Boston Scientific Scimed, Inc., Maple Grove, MN (US)

(72) Inventors: Yen-Hao Hsu, Shrewsbury, MA (US); Peijiang Wang, Brighton, MA (US)

(73) Assignee: Boston Scientific Scimed, Inc., Maple Grove, MN (US)

(21) Appl. No.: 19/214,553

(22) Filed: May 21, 2025

Related U.S. Application Data
(60) Provisional application No. 63/650,478, filed on May 22, 2024.

(51) Int. Cl. C08J 3/075; C08J 3/09; C08J 3/24; C08K 5/04

(52) U.S. Cl. CPC C08J 3/075; C08J 3/095; C08J 3/096; C08J 3/242; C08K 5/04; C08J 2300/106; C08J 2371/08

(57) ABSTRACT

In some aspects, the present disclosure pertains to a method of forming radiopaque crosslinked hydrogel particles comprising (a) mixing a reactive multi-arm polymer comprising a plurality of cyclic imide ester groups, a reactive multifunctional compound comprising a plurality of amino groups, and a radiocontrast agent under conditions in which a pH environment surrounding the reactive multi-arm polymer, the reactive multifunctional compound and the radiocontrast agent increases from an acidic pH to a basic pH, thereby forming a radiopaque hydrogel and (b) subjecting the hydrogel to a particle size reduction process, thereby forming the radiopaque crosslinked hydrogel particles. Other aspects of the present disclosure pertain to radiopaque crosslinked hydrogel particles formed from such methods, suspensions of such radiopaque crosslinked hydrogel particles, and medical supplies that contain such radiopaque crosslinked hydrogel particles.

### CROSS-REFERENCE TO RELATED APPLICATIONS

This application claims the benefit of U.S. Provisional Patent Application Ser. No. 63/650,478 filed on May 22, 2024, the disclosure of which is incorporated herein by reference.

### FIELD

The present disclosure relates to radiopaque crosslinked hydrogel particles, to injectable suspensions containing radiopaque crosslinked hydrogel particles, and to methods of making the same. The suspensions of radiopaque crosslinked hydrogel particles are useful in various medical applications.

### BACKGROUND

Bioresorbable hydrogels with rapid crosslinking reaction rate in vivo, known by the trade name of SpaceOAR®, have become a prominent biomaterial and obtained clinical success in creating the space between prostate and rectum, tremendously improving patient safety during the cancer therapies. A further improvement based on this application is that some of 8-Arm PEG branches are functionalized with 2,3,5-triiiodobenzamide (TIB) groups, replacing part of the activated ester end groups, succinimidyl glutarate (SG), in order to provide intrinsic radiopacity to the hydrogels themselves for CT-visibility. This hydrogel, known by the trade name of SpaceOAR Vue®, is the next generation of SpaceOAR® for prostate medical applications. The hydrogels break down in-vivo over the course of ca. 6-9 months. The breakdown occurs primarily through the hydrolysis of the ester linkages on the glutarate groups.

While the above approach is effectual, the entire functionalization process is complex, involving multiple steps, typically five steps, from commercially available hydroxyl-terminated 8-arm PEG to its functionalized form with two different end groups (TIB and SG groups), resulting in a significant increase of the product cost. Furthermore, the functionalized 8-arm PEG is co-injected with trilysine as a crosslinking agent, requiring the use of a double-barrel syringe.

For these and other reasons, alternative strategies for forming radiopaque injectable hydrogels are desired.

### SUMMARY

In some aspects, the present disclosure pertains to a method of forming radiopaque crosslinked hydrogel particles comprising (a) mixing a reactive multi-arm polymer comprising a plurality of cyclic imide ester groups, a reactive multifunctional compound comprising a plurality of amino groups, and a radiocontrast agent under conditions in which a pH environment surrounding the reactive multi-arm polymer, the reactive multifunctional compound and the radiocontrast agent increases from an acidic pH to a basic pH, thereby forming a radiopaque hydrogel and (b) subjecting the hydrogel to a particle size reduction process, thereby forming the radiopaque crosslinked hydrogel particles.

In some embodiments, a first solution that is buffered at a first pH ranging from 3.5 to 6.0 and contains the reactive multi-arm polymer, the reactive multifunctional compound, and the radiocontrast agent is mixed with a second solution that is buffered at a second pH ranging from 8.0 to 12.0.

In some embodiments, a first solution that is buffered at a first pH ranging from 3.5 to 6.0 and contains the reactive multi-arm polymer and the reactive multifunctional compound is mixed with a second solution that is buffered at a second pH ranging from 8.0 to 12.0 and contains the radiocontrast agent.

In some embodiments, which can be used in conjunction with any of the above aspects and embodiments, the mixing takes place in a planetary centrifugal mixer.

In some embodiments, which can be used in conjunction with any of the above aspects and embodiments, the particle size reduction process comprises processing the hydrogel in a homogenizer.

In some embodiments, which can be used in conjunction with any of the above aspects and embodiments, the method further comprises filtering and/or sieving the radiopaque crosslinked hydrogel particles after being subjected to the particle size reduction process.

In some embodiments, which can be used in conjunction with any of the above aspects and embodiments, the method further comprises filtering and drying the radiopaque crosslinked hydrogel particles after being subjected to the particle size reduction process.

In some embodiments, which can be used in conjunction with any of the above aspects and embodiments, the method further comprises suspending the radiopaque crosslinked hydrogel particles in a carrier fluid to form a hydrogel particle suspension.

In some embodiments, which can be used in conjunction with any of the above aspects and embodiments, the carrier fluid has a pH ranging from 3.5 to 6.0. In some of these embodiments, the carrier fluid comprises a linear polymer. For example, the linear polymer may be selected from a polyalkylene oxide linear polymer, a polyoxazoline linear polymer, a polypyrrolidone linear polymer, a polyacrylamide linear polymer, and a polyhydroxyethylmethacrylate linear polymer and/or the linear polymer may have a number average molecular weight ranging from 5 to 20 kDa.

In some embodiments, which can be used in conjunction with any of the above aspects and embodiments, the hydrogel particle suspension is placed into a container selected from a syringe barrel, a vial or an ampule. In some of these embodiments, the hydrogel particle suspension is sterilized before or after being placed into the container.

In some embodiments, which can be used in conjunction with any of the above aspects and embodiments, the hydrogel particle suspension is contained in a preloaded syringe. In some of these embodiments, the method further comprises packaging the preloaded syringe, a needle, and a luer connector in one or more sterile trays.

In some embodiments, which can be used in conjunction with any of the above aspects and embodiments, the radiocontrast agent is selected from non-ionic iodinated organic compounds and ionic iodinated organic compounds.

In some embodiments, which can be used in conjunction with any of the above aspects and embodiments, the multi-arm polymer comprises three or more polymer arms linked to a core region, each of the polymer arms comprising a hydrophilic polymer segment and cyclic imide ester end group. In some of these embodiments, each of the polymer arms comprises a hydrolysable ester group disposed between the hydrophilic polymer segment and the cyclic imide ester group and/or the hydrophilic polymer segment is selected from polyalkylene oxide segments, polyester segments, polyoxazoline segments, polydioxanone segments, and polypeptide segments and/or the core region comprises a polyol residue.

In some embodiments, which can be used in conjunction with any of the above aspects and embodiments, the reactive multifunctional compound is selected from a poly(amino acid) and a multi-arm amino-terminated PEG. In some of these embodiments, the reactive multifunctional compound is trilysine.

Potential benefits associated with the present disclosure include one or more of the following: radiocontrast is maintained or enhanced, a single syringe is used, injection volume is readily scalable, and there are no time constraints associated with injection of a composition that is in the process of becoming crosslinked while in the injection apparatus.

The above and other aspects, embodiments, features and benefits of the present disclosure will be readily apparent from the following detailed description.

### BRIEF DESCRIPTION OF THE DRAWINGS

FIG. 1 schematically illustrates a process of forming a radiopaque crosslinked hydrogel, in accordance with an embodiment of the present disclosure.

FIG. 2 schematically illustrates a catheter and a syringe that is loaded with a hydrogel particle suspension, in accordance with an embodiment of the present disclosure.

### DETAILED DESCRIPTION

In various aspects, the present disclosure pertains to suspensions of radiopaque crosslinked hydrogel particles in carrier fluids and to methods of forming the same.

The concentration of the radiopaque crosslinked hydrogel particles in the suspension may vary, but the suspension typically contains between 1 wt % and 25 wt % (e.g., ranging anywhere from 1 wt % to 2.5 wt % to 5 wt % to 10 wt % to 15 wt % to 20 wt % to 25 wt %) of the radiopaque crosslinked hydrogel particles (dry weight) relative to the total weight of the suspension, more typically between 2.5 wt % and 15 wt %

The radiopaque crosslinked hydrogel particles may vary in size and typically range between 10 and 1500 μm in longest dimension (e.g., diameter for a spherical particle, length for an elongate or rod-shaped particle, greatest width for a plate-like particle, etc.) (e.g., ranging anywhere from 10 μm to 25 μm to 50 μm to 100 μm to 250 μm to 500 μm to 1000 μm to 1500 μm).

The radiopaque crosslinked hydrogel particles of the present disclosure include crosslinked reaction products of (a) a reactive multi-arm polymer comprising a plurality of cyclic imide ester groups, (b) a reactive multifunctional compound comprising a plurality of amino groups, and (c) a radiocontrast agent.

Radiocontrast agents for use in the present disclosure include non-ionic and ionic iodinated organic compounds such as 1,3,5-triiodo-2,4,6-trishydroxymethylbenzene, iodixanol, iotrolan, ioversol, iopamidol, iomeprol, iobitridol, iohexol impurity J, metrizamide, ioxilan, iopentol, iopromide, diatrizoate salts such as diatrizoate sodium and diatrizoate and/or diatrizoate meglumine, ioxaglate salts such as ioxaglate sodium and/or ioxaglate meglumine, and iothalamate salts such as iothalamate sodium and iothalamate meglumine, among others. Radiocontrast agents for use in the present disclosure also include non-iodinated and/or inorganic radiocontrast agents such as barium sulfate particles and metallic particles, for example, particles of tantalum, tungsten, platinum, gold, rhenium, niobium, molybdenum, and their alloys, which metallic particles may be spherical or non-spherical.

Structures for several radiocontrast agents for use in the present disclosure are provided here:

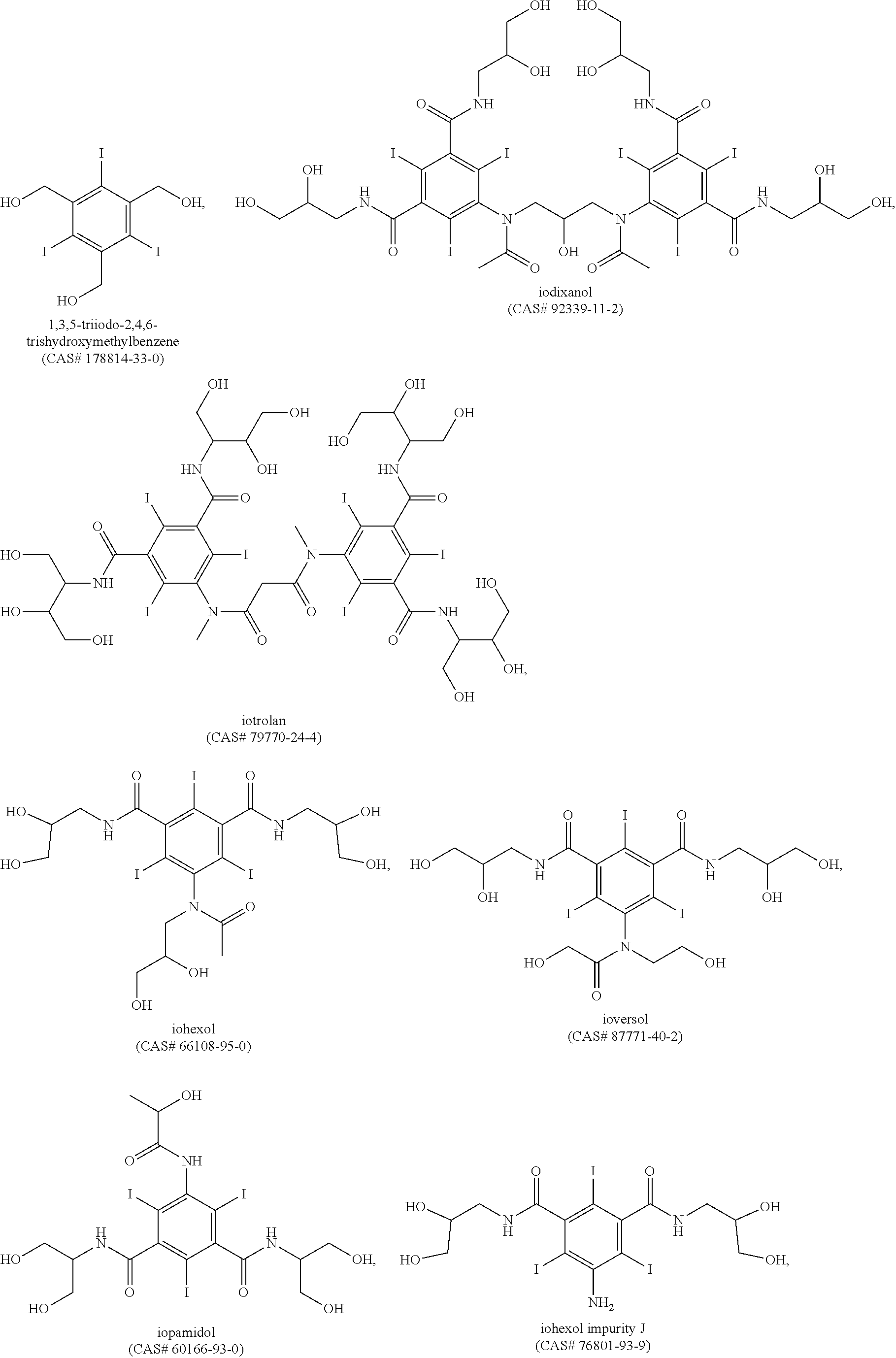

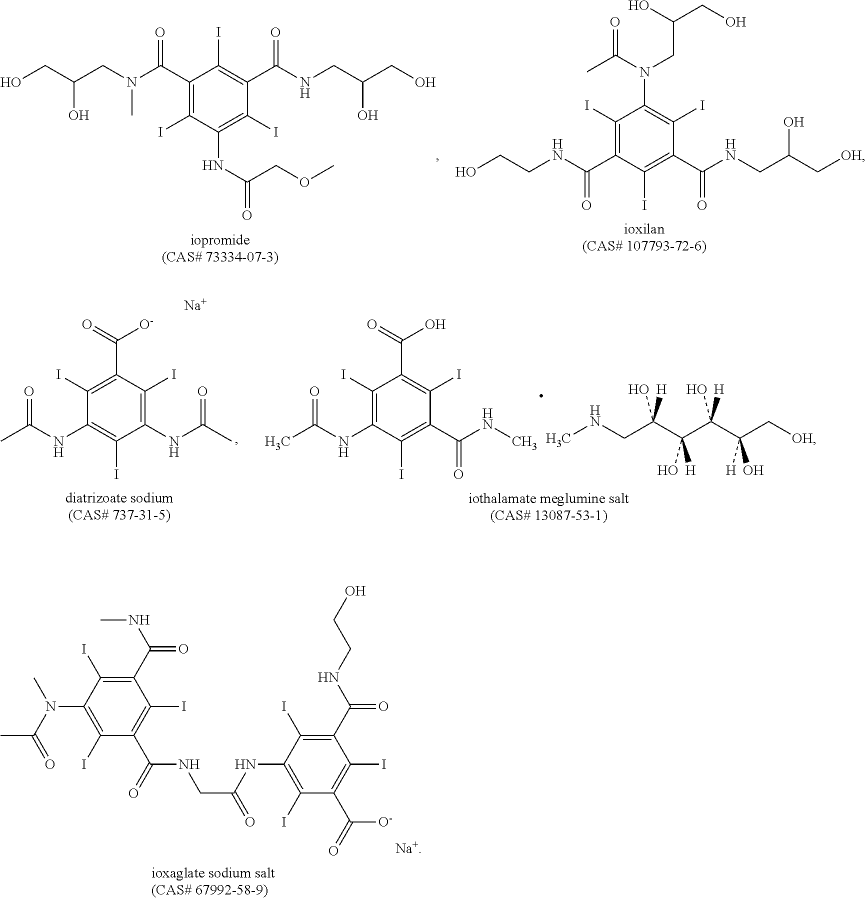

Reactive multi-arm polymers in accordance with the present disclosure include reactive multi-arm polymers that comprise a plurality of polymer arms linked to a core region, where the polymer arms comprise a hydrophilic polymer segment. One end of the hydrophilic polymer segment is covalently attached to the core region through a suitable linkage, and a cyclic imide ester group is covalently attached to an opposite end of the hydrophilic polymer segment through a suitable linkage.

Reactive multi-arm polymers in accordance with the present disclosure include polymers having from 2 to 100 arms, for example ranging anywhere from 2 to 3 to 4 to 5 to 6 to 7 to 8 to 9 to 10 to 11 to 12 to 15 to 20 to 25 to 50 to 75 to 100 arms (in other words, having a number of arms ranging between any two of the preceding values).

The cyclic imide ester groups may be linked to the hydrophilic polymer segment and the hydrophilic polymer segment may be linked to the core through any suitable linking moiety, which may be selected, for example, from a bond, a linking moiety that comprises an alkyl group, a linking moiety that comprises an ether group, a linking moiety that comprises an ester group, a linking moiety that comprises an amide group, a linking moiety that comprises an amine group, a linking moiety that comprises a carbonate group, a linking moiety that comprises a urethane group, a linking moiety that comprises a urea group, a linking moiety that comprises a ketone group, a linking moiety that comprises a triazole group, group, or a linking moiety that comprises a combination of two or more of any of the foregoing groups, among others. In various embodiments, the linking moiety comprises a hydrolysable ester group.

Hydrophilic polymer segments can be selected from any of a variety of synthetic, natural, or hybrid synthetic-natural hydrophilic polymer segments. Examples of hydrophilic polymer segments include those that are formed from one or more hydrophilic monomers selected from the following: C₁-C₆-alkylene oxides (e.g., ethylene oxide, propylene oxide, tetramethylene oxide, etc.), polar aprotic vinyl monomers (e.g. N-vinyl pyrrolidone, acrylamide, N-methyl acrylamide, dimethyl acrylamide, N-vinyl imidazole, 4-vinylimidazole, sodium 4-vinylbenzenesulfonate, etc.), dioxanone, ester monomers (e.g. glycolide, lactide, β-propiolactone, β-butyrolactone, γ-butyrolactone, γ-valerolactone, δ-valerolactone, δ-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2- oxazoline, 2-isobutyl-2-oxazoline, 2-hexyl-2-oxazoline, etc.), 2-phenyl-2-oxazoline, N-isopropylacrylamide, amino acids and sugars.

Hydrophilic polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(δ-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, polypeptide segments, and polysaccharide segments. Polysaccharide segments include those that contain one or more uronic acid species, such as galacturonic acid, glucuronic acid and/or iduronic acid, with particular examples of polysaccharide segments including alginic acid, hyaluronic acid, pectin, agaropectin, carrageenan, gellan gum, gum arabic, guar gum, xanthan gum, and carboxymethyl cellulose moieties.

Polymer segments for use in the multi-arm polymers of the present disclosure typically contain from 10 monomer units or less to 1000 monomer units or more, for example, ranging anywhere from 5 to 10 to 20 to 50 to 100 to 200 to 500 to 1000 to 2000 monomer units.

In certain embodiments, the core region comprises a residue of a polyhydroxy compound comprising two or more hydroxyl groups, also referred to herein as a polyol. Polyols for use in the present disclosure may have two, three, four, five, six, seven, eight, nine, ten, eleven, twelve, thirteen, fourteen, fifteen, sixteen, seventeen, eighteen, nineteen, twenty, twenty-five, thirty or more hydroxyl groups. In various embodiments, polyols for use in the present disclosure may have further hydrophilic groups in addition to hydroxyl groups, including ether groups, amine groups, ester groups, amide groups.

Polyols for use in the present disclosure include sugars (monosaccharides, disaccharides, trisaccharides, etc.), sugar alcohols, calixarenes, cyclodextrins, polyhydroxylated polymers, catechins, flavanols, anthocyanins, stilbenes, and polyphenols, among others.

Polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols. Specific examples include methane triol, glycerol, trimethylolpropane, benzenetriol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, catechins, flavanols, anthocyanins, stilbenes, polyphenols, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl) ethane, and 2,6-bis(hydroxyalkyl) cresols, among others.

Illustrative polyols also include polyhydroxylated polymers such as poly(vinyl alcohol), poly(allyl alcohol), poly(hydroxyethyl acrylate), or poly(hydroxyethyl methacrylate), among others. Such polyhydroxylated polymers may range, for example, from 2 to 100 monomer units in length.

Reactive multi-arm polymers in accordance with the present disclosure can be formed from hydroxy-terminated multi-arm polymers having arms that comprise one or more hydroxyl end groups. In some embodiments of the present disclosure, a polyol such as one of those described above, among others, may be used as a multi-functional initiator for polymer chain growth. For example, the polyol may be used as an initiator for ring-opening polymerization of ethylene oxide to form polyethylene oxide (PEO) segments (also referred to a polyethylene glycol, or PEG, segments) at each of the hydroxyl groups of the polyol. The resulting hydroxyl-terminated PEG segments possess tunable hydrophilicity depending on the desired water-solubility of the resulting multi-arm polymer, for example, with increasing PEG segment length leading to increasing hydrophilicity. Hydroxyl-terminated multi-arm polymers are also available commercially. For example, hydroxyl-terminated four-arm PEG, hydroxyl-terminated six-arm PEG, and hydroxyl-terminated eight-arm PEG are available from JenKem Technology USA, Plano, TX, U.S.A.

In some embodiments, a hydroxy-terminated multi-arm hydrophilic polymer may be reacted with a cyclic anhydride to form a carboxylic-acid-terminated polymer in which carboxylic acid end groups are linked to hydrophilic polymer segments through hydrolysable ester groups. For example, terminal hydroxyl groups of hydrophilic polymer segments may be reacted with a cyclic anhydride (e.g., glutaric anhydride, succinic anhydride, malonic anhydride, adipic anhydride, diglycolic anhydride, etc.) to form a carboxylic-acid-terminated segment such as a glutaric-acid-terminated segment, a succinic-acid-terminated segment, a malonic-acid-terminated segment, an adipic-acid-terminated segment, a diglycolic-acid-terminated segment, and so forth.

The preceding cyclic anhydrides, among others, may be reacted with a hydroxy-terminated multi-arm hydrophilic polymer under basic conditions to form a carboxylic-acid-terminated multi-arm hydrophilic polymer comprising a carboxylic acid end group that is linked to a hydrophilic polymer segment through a hydrolysable ester group. Carboxylic-acid-terminated multi-arm polymers are also available commercially. For example, carboxylic-acid-terminated four-arm PEG and carboxylic-acid-terminated eight-arm PEG (without hydrolysable ester groups) are available from JenKem Technology USA.

A cyclic imide ester group may then be linked to the carboxylic-acid-terminated multi-arm hydrophilic polymer. For instance, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic-acid-terminated multi-arm hydrophilic polymer in the presence of a suitable coupling agent (e.g., a carbodiimide coupling agent) to form an activated ester group, in particular, a cyclic imide ester group (e.g., an succinimide ester group, an maleimide ester group, an glutarimide ester group, an phthalimide ester group, a diglycolimide ester group, bicyclo[2.2.1]hept-5-ene-2,3-dicarboxylic acid imide ester group, etc.) that is linked to a hydrophilic polymer segment through a hydrolysable ester group. In this way, a number of reactive diester groups may be formed. For example, in the particular case of N-hydroxysuccinimide as an N-hydroxy cyclic imide compound, exemplary reactive diester groups include succinimidyl malonate groups, succinimidyl glutarate groups, succinimidyl succinate groups, succinimidyl adipate groups, and succinimidyl diglycolate groups, among others. In the particular case of HONB as an N-hydroxy cyclic imide compound, exemplary reactive diester groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, and bicyclo[2.2.1]hept-5-ene-2,3-dicarboxylic acid imidyl diglycolate groups, among others. In the particular case of N-hydroxymaleimide as an N-hydroxy cyclic imide compound, exemplary reactive diester groups include maleimidyl malonate groups, maleimidyl glutarate groups, maleimidyl succinate groups, maleimidyl adipate groups, and maleimidyl diglycolate groups, among others. In the particular case of N-hydroxyglutarimide as an N-hydroxy cyclic imide compound, exemplary reactive diester groups include glutarimidyl malonate groups, glutarimidyl glutarate groups, glutarimidyl succinate groups, glutarimidyl adipate groups, glutarimidyl diglycolate groups, among others. In the particular case of N-hydroxyphthalimide as an N-hydroxy cyclic imide compound, exemplary reactive diester groups include phthalimidyl malonate groups, phthalimidyl glutarate groups, phthalimidyl succinate groups, phthalimidyl adipate groups, and phthalimidyl diglycolate groups, among others. Some multi-arm polymers having reactive diester end groups are also available commercially. For example, succinimidyl-glutarate-terminated four-arm PEG and succinimidyl-glutarate-terminated eight-arm PEG are available from JenKem Technology USA.

As previously noted, radiopaque crosslinked hydrogel particles of the present disclosure include crosslinked reaction products of (a) a reactive multi-arm polymer comprising a plurality of cyclic imide ester groups, (b) a reactive multifunctional compound comprising a plurality of amino groups, and (c) a radiocontrast agent.

Reactive multifunctional compound comprising a plurality of amino groups, also referred to herein as polyamino compounds, suitable for use in the present disclosure include, for example, small molecule polyamines (e.g., containing at least two amine groups, for example ranging anywhere from 2 to 3 to 4 to 5 to 6 to 7 to 8 to 9 to 10 to 11 to 12 to 15 to 20 amine groups or more in certain embodiments), polymers having amine side groups, and branched polymers having amine end groups, including dendritic polymers having amine end groups. Polyamino compounds suitable for use in the present disclosure include those that comprises a plurality of —(CH₂)ₓ—NH₂ groups where x is 0, 1, 2, 3, 4, 5 or 6.

Polyamino compounds suitable for use in the present disclosure include polyamino compounds that comprise two or more basic amino acid residues, including residues of amino acids having primary amine groups, such as lysine and ornithine, for example, polyamines that comprise from 2 to 20 lysine and/or ornithine amino acid residues (e.g., polylysine compounds such as dilysine, trilysine, tetralysine, pentalysine, hexalysine, etc., polyornithine compounds such as diornithine, triornithine, tetraornithine, pentaornithine, hexaornithine, etc., and poly(lysine-co-ornithine) compounds).

Particular examples of polyamino compounds which may be used as the multifunctional compound further include ethylenetriamine, diethylene triamine, hexamethylenetriiamine, di(heptamethylene) triamine, di(trimethylene) triamine, bis(hexamethylene) triamine, triethylene tetramine, tripropylene tetramine, tetraethylene pentamine, hexamethylene heptamine, pentaethylene hexamine, dimethyl octylamine, dimethyl decylamine, and JEFFAMINE polyetheramines available from Huntsman Corporation, chitosan and derivatives thereof, poly(vinyl amine), and poly(allyl amine), among others among others.

A radiopaque hydrogel may be provided by mixing (a) a first solution (also referred to herein as a diluent solution) of a reactive multi-arm polymer as described herein and a polyamino compound as described herein and (b) a second solution (also referred to herein as an accelerant solution) that contains a radiopaque compound as described herein and acts to accelerate a crosslinking reaction between the reactive multi-arm polymer and the reactive polyamino compound. In various embodiments, the first solution, the second solution, or both, further contain a radiocontrast agent.

In some embodiments, the first solution is buffered to an acidic pH, for example, having a pH ranging from 3 to 6.5, more typically ranging from 3.8 to 4.2. Such acidic pH conditions act to suppress crosslinking between the reactive multi-arm polymer and the reactive multifunctional compound, thereby preventing the reactive multi-arm polymer and the reactive multifunctional compound from crosslinking prematurely. Such acidic pH conditions may be provided using any suitable buffer such as Sodium Phosphate Monobasic and/or MES (2-(N-morpholino) ethanesulfonic acid). The concentration of the reactive multi-arm polymer in the first solution may range, for example, from 10% to 25%, more typically, ranging from 14% to 22%. The concentration of the reactive multifunctional compound in the first solution may range, for example, from 0.4% to 1%, more typically, ranging from 0.5% to 0.75%. Where the first solution contains a radiocontrast agent, a concentration of the radiocontrast agent may range, for example, from 1% to 5%.

In some embodiments, the second solution is buffered to a basic pH, for example, having a pH ranging from 8 to 12, more typically ranging from 9 to 11. Such basic pH conditions act to accelerate crosslinking between the reactive multi-arm polymer and the reactive multifunctional compound. Such basic pH conditions may be provided using any suitable buffer such as Sodium Tetraborate Decahydrate and/or Sodium Phosphate Dibasic. Where the second solution contains a radiocontrast agent, a concentration of the radiocontrast agent may range, for example, from 1% to 5%.

Mixing the first and second solutions results in crosslinking between the reactive multi-arm polymer and the reactive multifunctional compound and the formation of a hydrogel in which the radiocontrast agent is dispersed throughout the hydrogel. The radiocontrast agent is trapped within a crosslinked network that is formed upon reaction of the reactive multi-arm polymer and the reactive multifunctional compound.

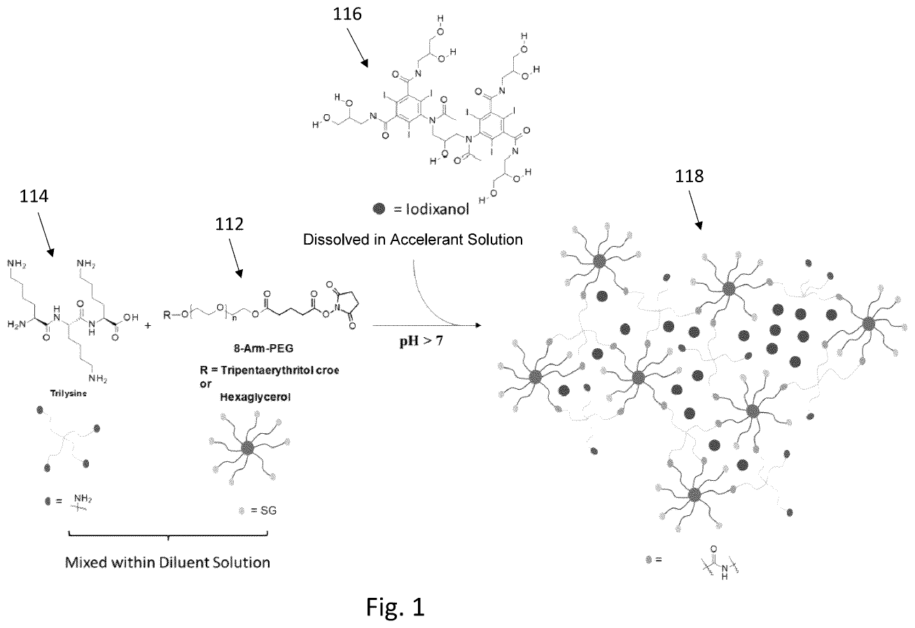

Turning now to FIG. 1, a diluent solution may be provided that contains 8-arm polyethylene oxide 112 and trilysine 114, wherein the multi-arm polyethylene oxide 112 and trilysine 114 are prevented from crosslinking with one another to any significant degree by maintaining the pH at an acidic value, for example, using a suitable acidic buffer. With regard to the 8-arm polyethylene oxide 112, n may have a value ranging, for example, from 22 to 100, and R represents a core, which may be, for example, a residue of a polyol such as hexaglycerol or tripentaerythritol. Also, although only one arm of the 8-arm polyethylene oxide 112 is shown attached to the core R, it is to be understood that additional polymer arms are present. A crosslinked hydrogel 118 is formed by mixing the diluent solution with an accelerant solution that contains a radiocontrast agent, specifically, dissolved iodixanol 116, and is buffered to a basic pH.

The hydrogel may then be processed into particles of suitable size. Particles may be formed using any suitable process, for instance by homogenization, grinding (including cryogrinding), crushing, milling, pounding, forcing through a screen or the like. Homogenizers that can be used for this purpose include rotor-stator homogenizers, high-pressure homogenizers, ultrasonic homogenizers, bead mill homogenizers, and piston homogenizers, among others.

Sieving, filtering, or other known techniques may be used in some embodiments to classify and fractionate the particles. Crosslinked hydrogel particles formed using the above and other techniques may vary widely in size, for example, having an average size ranging from 50 to 950 microns.

Hydrogel particle suspensions in accordance with the present disclosure further include a carrier fluid. The carrier fluid contains water, which may be, for example, in the form of water for injection, saline, or phosphate buffered saline. The concentration of water in the carrier fluid may vary but is typically more than 65 wt %, more typically more than 85 wt %, and in some embodiments more than 90 wt %, relative to the total weight of the carrier fluid.

In some embodiments, the carrier fluid contains a buffer that can, for example, stabilize the particles, maintain gel longevity and/or reduce required injection force. For example, in some embodiments, the carrier fluid includes an acidic buffer and has a pH ranging from 3 to 6.5, for example, ranging from 3.5 to 4.5. Generally, any buffer that is biocompatible and suitable for injection may be employed, with monobasic sodium phosphate being one particular example.

In some embodiments, the carrier fluid further contains one or more types of linear hydrophilic polymer that act as a lubricant and significantly improve the injectability of the radiopaque crosslinked hydrogel particles by reducing injection force.

Linear hydrophilic polymers in accordance with the present disclosure can be selected from any of a variety of synthetic, natural, or hybrid synthetic-natural hydrophilic polymers. Examples of linear hydrophilic polymers include linear homopolymers and linear copolymers formed from one or more of the following hydrophilic monomers: ethylene oxide, propylene oxide, N-vinyl pyrrolidone, oxazoline monomers (e.g., 2-alkyl-2-oxazolines, for instance, 2-(C₁-C₆ alkyl)-2-oxazolines, including various isomers, such as 2-methyl-2-oxazoline, 2-ethyl-2-oxazoline, 2-propyl-2-oxazoline, 2-isopropyl-2-oxazoline, 2-n-butyl-2-oxazoline, 2-n-hexyl-2-oxazoline, etc., and 2-phenyl-2-oxazoline), vinyl alcohol, allyl alcohol, hydroxyethyl acrylate, hydroxyethyl methacrylate, acrylamide, N-isopropylacrylamide, and amino acids.

Linear hydrophilic polymers may be selected, for example, from one or combination of any of the following homopolymers and copolymers: polyethers including poly(alkylene oxides) such as poly(ethylene oxide) (PEO) (also referred to as polyethylene glycol or PEG), poly(propylene oxide), poly(ethylene oxide-co-propylene oxide), poly(N-vinyl pyrrolidone), poly-2-oxazolines including poly(2-C₁-C₆-alkyl-2-oxazoline)s such as poly(2-methyl-2-oxazoline), poly(2-ethyl-2-oxazoline), poly(2-propyl-2-oxazoline), poly(2-isopropyl-2-oxazoline), and poly(2-n-butyl-2-oxazoline), poly(2-phenyl-2-oxazoline), poly(vinyl alcohol), poly(allyl alcohol), polyhydroxyethyl acrylate, polyhydroxyethyl methacrylate, polyacrylamide and derivatives of the same, such as poly(N-isopropylacrylamide), and polypeptides.

In some embodiments, the linear hydrophilic polymer will have the same monomer composition as the arms of the reactive multi-arm polymer that is used to form the hydrogel particles.

In certain embodiments, the linear hydrophilic polymer may be selected from poly(ethylene oxide), poly-2-oxazolines (which have various side groups that can tune the hydrophilicity of the same), polyvinylpyrrolidone, polyacrylamide, poly(N-isopropylacrylamide), or a polypeptide.

Linear hydrophilic polymers in accordance with the present disclosure may vary in length but typically have a weight average molecular weight (M_{w}) ranging from 1 kDa to 60 kDa, for example, ranging anywhere from 1 kDa to 2 kDa to 5 kDa to 10 kDa to 15 kDa to 20 kDa to 30 kDa to 40 kDa to 50 kDa to 60 kDa (i.e., between any two of the preceding values), among other possible values.

The concentration of the one or more types of linear hydrophilic polymers in the carrier fluid may vary but typically contains between 1 wt % and 35 wt % relative to the weight of the carrier fluid, for example, ranging anywhere from 1 wt % to 2.5 wt % to 5 wt % to 10 wt % to 15 wt % to 20 wt % to 25 wt % to 30 wt % to 35 wt %.

In some embodiments, the hydrogel particle suspensions of the present disclosure may contain one or more additional agents. Examples of such additional agents include therapeutic agents, imaging agents, colorants, tonicity adjusting agents, suspension agents and wetting agents.

Examples of therapeutic agents include antithrombotic agents, anticoagulant agents, antiplatelet agents, thrombolytic agents, anti-cancer drugs, antiproliferative agents, anti-inflammatory agents, hyperplasia inhibiting agents, anti-restenosis agents, steroids, anti-allergic agents, hemostatic agents, smooth muscle cell inhibitors, antibiotics, antimicrobials, anti-fungal agents, analgesics, anesthetics, immunosuppressants, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, and STING (stimulator of interferon genes) agonists, among others.

Examples of imaging agents, other than the radiocontrast agents described herein, include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogel particle suspensions of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, and (e) imageable radioisotopes including 99mTc, 201Th, 51Cr, 67Ga, 68Ga, 111 In, 64Cu, 89Zr, 59Fe, 42K, 82Rb, 24Na, 45Ti, 44Sc, 51Cr and 177Lu, among others.

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants and wetting agents.

In various embodiments, the hydrogel particle suspensions of the present disclosure are visible under fluoroscopy. In various embodiments, the hydrogel particle suspensions have a radiopacity that is greater than 100 Hounsfield units (HU), beneficially anywhere ranging from 100 HU to 250 HU to 500 HU to 750 HU to 1000 HU to 2000 HU or more (in other words, ranging between any two of the preceding numerical values).

The hydrogel particle suspensions may be shipped, for example, in a syringe, catheter, vial, ampoule, or other suitable container. The hydrogel particle suspensions of the present disclosure may be stored and transported in a sterile form. The hydrogel particle suspensions may be sterilized before or after being placed in the container. The hydrogel particle suspensions herein may be sterilized using any suitable method. For example, the hydrogel particle suspensions may be sterilized by heating the mixture at or to a temperature of about 121° C. Alternatively or additionally, the compositions may be sterilized via sterile filtration and/or by supercritical CO₂, gamma ray, x-ray or electron beam irradiation.

In various embodiments, kits are provided that include one or more delivery devices for delivering the hydrogel particle suspensions to a subject. Such kits may include one or more of the following elements: a syringe barrel, which may or may not contain a carrier fluid and/or radiopaque crosslinked hydrogel particles as described herein; a vial, which may or may not contain a carrier fluid and/or radiopaque crosslinked hydrogel particles as described herein; a needle; luer connector; a flexible tube (e.g., a catheter adapted to fluidly connect the needle to the syringe); and an injectable liquid such as water for injection, normal saline or phosphate buffered saline. The carrier fluid will typically be provided in a syringe or a vial. Whether supplied in a syringe, vial, or other reservoir, the radiopaque crosslinked hydrogel particles may be provided in a form that is ready for injection (e.g., as a suspension of the radiopaque crosslinked hydrogel particles in the carrier fluid) or may be provided in dry form (e.g., powder form) which can be combined with a suitable fluid to form a suspension. The kit elements may be provided in one or more sterile trays.

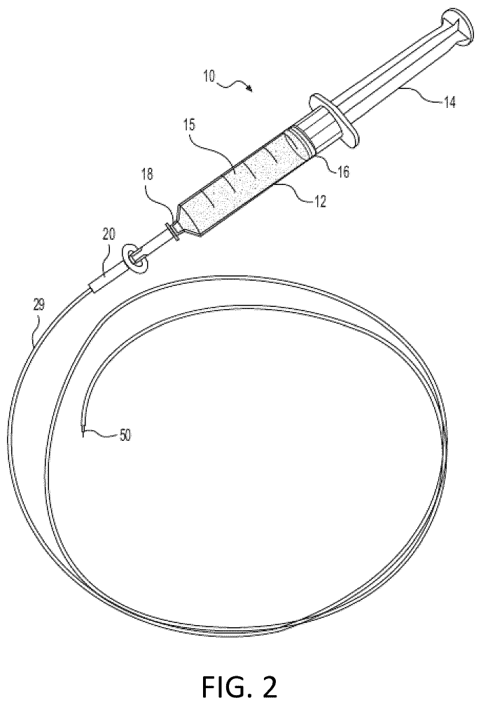

For example, FIG. 2 illustrates an exemplary syringe 10 providing a reservoir for a hydrogel particle suspension 15 as described herein. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to an injection needle 50. The syringe barrel 12 may serve as a reservoir containing the hydrogel particle suspension 15 for injection through the needle 50.

The hydrogel particle suspensions described herein can be used for a number of purposes.

For example, hydrogel particle suspensions can be injected to provide spacing between tissues, hydrogel particle suspensions can be injected (e.g., in the form of blebs) to provide fiducial markers, hydrogel particle suspensions can be injected for tissue augmentation or regeneration, hydrogel particle suspensions can be injected as a filler or replacement for soft tissue, hydrogel particle suspensions can be injected to provide mechanical support for compromised tissue, hydrogel particle suspensions be injected as a scaffold, and/or hydrogel particle suspensions can be injected as a carrier of therapeutic agents in the treatment of diseases and cancers and the repair and regeneration of tissue, among other uses.

The hydrogel particle suspensions of the present disclosure may be used in a variety of medical procedures, including the following, among others: a procedure to implant a fiducial marker comprising a radiopaque crosslinked hydrogel, a procedure to implant a tissue regeneration scaffold comprising a radiopaque crosslinked hydrogel, a procedure to implant a tissue support comprising a radiopaque crosslinked hydrogel, a procedure to implant a tissue bulking agent comprising a radiopaque crosslinked hydrogel, a procedure to implant a therapeutic-agent-containing depot comprising a radiopaque crosslinked hydrogel, a tissue augmentation procedure comprising implanting a radiopaque crosslinked hydrogel, a procedure to introduce a radiopaque crosslinked hydrogel between a first tissue and a second tissue to space the first tissue from the second tissue.

The hydrogel particle suspensions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intra-vitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

### EXAMPLES

Several exemplary embodiments follow.

It is noted that for Examples 1, 4 and 6, other commercially available liquid radiocontrast agents can be used in place of Visipaque™, including other non-ionic liquid radiocontrast agents such as Omnipaque™ iohexol solution, Ultravist® iopromide solution, Optiray® ioversol solution, Oxilan,® ioxilan solution, and Iosovue® iopamidol solution, or ionic liquid radiocontrast agents such as Hypaque™ (diatrizoate sodium), Hexabrix™ (ioxaglate meglumine and ioxaglate sodium), and Cysto-Conray® (Iothalamate meglumine), as well as non-iodinated contrast media such as VoLumen® barium sulfate suspension, among others.

Also, while iodixanol in solid form (e.g., powder form) is used to form solutions in Examples 2, 3, 5 and 6, other commercially available solid radiocontrast agents can be used, including non-ionic radiocontrast agents such as iohexol, iopromide, ioversol, ioxilan, ionic radiocontrast agents such as diatrizoate, ioxaglate or iothalamate salts, and non-iodinated radiocontrast agents such as barium sulfate, gold nanoparticles, and so forth.

### Example 1

The following are prepared: (a) first reservoir containing a multi-arm hydrophilic polymer having multiple cyclic imide ester groups, for example, succinimidyl-glutarate-terminated eight-arm PEG, in a suitable solid form, such as a powder form (0.9 g to 1.8 g), (b) a second reservoir containing 250 μL to 500 μL of a liquid radiocontrast agent such as Visipaque™, an isotonic, aqueous solution containing iodixanol available from GE HealthCare, Chicago, IL, USA, and a polyamine crosslinker such as trilysine (60 mg to 120 mg) dissolved in an aqueous buffered diluent solution having a pH ranging from 3.5 to 6.0, more typically from 3.8 to 4.2, that comprises water and a suitable buffering agent, such as monobasic sodium phosphate (6 mL total volume), and (c) a third reservoir containing an aqueous buffered accelerant solution having a pH ranging from 8.0 to 12.0, more typically 9.0 to 11.0, that comprises water and a suitable buffering agent, such as sodium borate and dibasic sodium phosphate (6 mL total volume). The contents of the first reservoir are combined with the contents of the second reservoir using a suitable mixer (e.g., a planetary centrifugal mixer, such as a planetary centrifugal mixer available from THINKY U.S.A., Inc., Laguna Hills, CA, USA) to form a precursor solution that is buffered to an acidic pH and contains the multi-arm hydrophilic polymer, the polyamine and the radiocontrast agent. This precursor solution is then mixed with the accelerant solution in the third reservoir using a suitable mixer (e.g., a planetary centrifugal mixer), thereby forming a radiocontrast-agent-containing hydrogel.

### Example 2

The following are prepared: (a) first reservoir containing a multi-arm hydrophilic polymer having multiple cyclic imide ester groups, for example, succinimidyl-glutarate-terminated eight-arm PEG, in a suitable solid form, such as a powder form (0.9 g to 1.8 g), (b) a second reservoir containing a radiocontrast agent such as iodixanol (10 mg to 50 mg) and a polyamine crosslinker such as trilysine (60 mg to 120 mg) dissolved in an aqueous buffered diluent solution having a pH ranging from 3.5 to 6.0, more typically from 3.8 to 4.2, that comprises water and a suitable buffering agent, such as monobasic sodium phosphate (6 mL total volume), and (c) a third reservoir containing an aqueous buffered accelerant solution having a pH ranging from 8.0 to 12.0, more typically 9.0 to 11.0, that comprises water and a suitable buffering agent, such as sodium borate and dibasic sodium phosphate (6 mL total volume). The contents of the first reservoir are combined with the contents of the second reservoir using a suitable mixer (e.g., a planetary centrifugal mixer) to form a precursor solution that is buffered to an acidic pH and contains the multi-arm hydrophilic polymer, the polyamine and the radiocontrast agent. This precursor solution is then mixed with the accelerant solution in the third reservoir using a suitable mixer (e.g., a planetary centrifugal mixer), thereby forming a radiocontrast-agent-containing hydrogel.

### Example 3

The following are prepared: (a) first reservoir containing a multi-arm hydrophilic polymer having multiple cyclic imide ester groups, for example, succinimidyl-glutarate-terminated eight-arm PEG, in a suitable solid form such as a powder form (0.9 g to 1.8 g) and a radiocontrast agent such as iodixanol in a suitable solid form such as a powder form (10 mg to 50 mg), (b) a second reservoir containing a polyamine crosslinker such as trilysine (60 mg to 120 mg) dissolved in an aqueous buffered diluent solution having a pH ranging from 3.5 to 6.0, more typically from 3.8 to 4.2, that comprises water and a suitable buffering agent, such as monobasic sodium phosphate (6 mL total volume), and (c) a third reservoir containing an aqueous buffered accelerant solution having a pH ranging from 8.0 to 12.0, more typically 9.0 to 11.0, that comprises water and a suitable buffering agent, such as sodium borate and dibasic sodium phosphate (6 mL total volume). The contents of the first reservoir are combined with the contents of the second reservoir using a suitable mixer (e.g., a planetary centrifugal mixer) to form a precursor solution that is buffered to an acidic pH and contains the multi-arm hydrophilic polymer, the polyamine and the radiocontrast agent. This precursor solution is then mixed with the accelerant solution in the third reservoir using a suitable mixer (e.g., a planetary centrifugal mixer), thereby forming a radiocontrast-agent-containing hydrogel.

### Example 4

The following are prepared: (a) first reservoir containing a multi-arm hydrophilic polymer having multiple cyclic imide ester groups, for example, succinimidyl-glutarate-terminated eight-arm PEG, in a suitable solid form such as a powder form (0.9 g to 1.8 g), (b) a second reservoir containing a polyamine crosslinker such as trilysine (60 mg to 120 mg) dissolved in an aqueous buffered diluent solution having a pH ranging from 3.5 to 6.0, more typically from 3.8 to 4.2, that comprises water and a suitable buffering agent, such as monobasic sodium phosphate (6 mL total volume), and (c) a third reservoir containing 250 μL to 500 μL of a liquid radiocontrast agent such as Visipaque™ dissolved in an aqueous buffered accelerant solution having a pH ranging from 8.0 to 12.0, more typically 9.0 to 11.0, that comprises water and a suitable buffering agent, such as sodium borate and dibasic sodium phosphate (6 mL total volume). The contents of the first reservoir are combined with the contents of the second reservoir using a suitable mixer (e.g., a planetary centrifugal mixer) to form a precursor solution that is buffered to an acidic pH and contains the multi-arm hydrophilic polymer and the polyamine. This precursor solution is then mixed with the accelerant solution containing the radiocontrast agent in the third reservoir using a suitable mixer (e.g., a planetary centrifugal mixer), thereby forming a radiocontrast-agent-containing hydrogel.

### Example 5

The following are prepared: (a) first reservoir containing a multi-arm hydrophilic polymer having multiple cyclic imide ester groups, for example, succinimidyl-glutarate-terminated eight-arm PEG, in a suitable solid form such as a powder form (0.9 g to 1.8 g), (b) a second reservoir containing a polyamine crosslinker such as trilysine (60 mg to 120 mg) dissolved in an aqueous buffered diluent solution having a pH ranging from 3.5 to 6.0, more typically from 3.8 to 4.2, that comprises water and a suitable buffering agent, such as monobasic sodium phosphate (6 mL total volume), and (c) a third reservoir containing a radiocontrast agent such as iodixanol (10 mg to 50 mg) dissolved in an aqueous buffered accelerant solution having a pH ranging from 8.0 to 12.0, more typically 9.0 to 11.0, that comprises water and a suitable buffering agent, such as sodium borate and dibasic sodium phosphate (6 mL total volume). The contents of the first reservoir are combined with the contents of the second reservoir using a suitable mixer (e.g., a planetary centrifugal mixer) to form a precursor solution that is buffered to an acidic pH and contains the multi-arm hydrophilic polymer and the polyamine. This precursor solution is then mixed with the accelerant solution containing the radiocontrast agent in the third reservoir using a suitable mixer (e.g., a planetary centrifugal mixer), thereby forming a radiocontrast-agent-containing hydrogel.

### Example 6

The following are prepared: (a) first reservoir containing a multi-arm hydrophilic polymer having multiple cyclic imide ester groups, for example, succinimidyl-glutarate-terminated eight-arm PEG, in a suitable solid form such as a powder form (0.9 g to 1.8 g), (b) a second reservoir containing a polyamine crosslinker such as trilysine (60 mg to 120 mg) dissolved in an aqueous buffered diluent solution having a pH ranging from 3.5 to 6.0, more typically from 3.8 to 4.2, that comprises water and a suitable buffering agent, such as monobasic sodium phosphate (4 mL total volume), (c) a third reservoir containing a radiocontrast agent such as iodixanol in a suitable solid form such as a powder form (10 mg to 50 mg) dissolved in an aqueous buffered accelerant solution having a pH ranging from 8.0 to 12.0, more typically 9.0 to 11.0, that comprises water and a suitable buffering agent, such as sodium borate and dibasic sodium phosphate (4 mL total volume), and (d) a fourth reservoir containing water and either a radiocontrast agent in liquid form such as Visipaque™ (250 μL to 500 μL) or a radiocontrast agent such as iodixanol in a suitable solid form such as a powder form (10 mg to 50 mg) (4 mL total volume) dissolved in the water. The contents of the first reservoir, the second reservoir, and the fourth reservoir are mixed in any order using a suitable mixer (e.g., a planetary centrifugal mixer) to form a precursor solution that is buffered to an acidic pH and contains the multi-arm hydrophilic polymer, the polyamine, and the radiocontrast agent. For example, the precursor solution may be formed in any of the following ways: (a) the contents of the first, second and fourth reservoirs can be mixed simultaneously, (b) the contents of the first and second reservoirs can be first mixed after which the contents of the fourth reservoir are mixed with the mixture of the contents from the first and second reservoirs, (c) the contents of the first and fourth reservoirs can be first mixed after which the contents of the second reservoir are mixed with the mixture of the contents from the first and fourth reservoirs, or (d) the contents of the second and fourth reservoirs can be first mixed after which the contents of the first reservoir are mixed with the mixture of the contents from the second and fourth reservoirs. This precursor solution is then mixed with the accelerant solution in the third reservoir using a suitable mixer (e.g., a planetary centrifugal mixer), thereby forming a radiocontrast-agent-containing hydrogel.

### Example 7

A radiocontrast-agent-containing hydrogel formed in accordance with any of Examples 1-6 is broken down into hydrogel particles using a suitable technique such as processing in a rotor-stator homogenizer. Once the desired particle size is achieved (e.g., particles sized between 50 and 800 microns), the hydrogel particles are then washed and dried using a suitable process such as a Nutsche filtration and drying process. Washing can be performed with an aqueous buffered diluent solution like that of Example 1. The dried hydrogel particles are then reformulated as a suspension, for example, a buffered suspension containing the hydrogel particles, a suitable buffering agent to provide a pH ranging from 3.5 to 6.0, more typically from 3.8 to 4.2, such as such as monobasic sodium phosphate, and a linear hydrophilic polymer, such as linear PEG, that acts as a lubricant and reduces injection force. For example, the buffered suspension may contain from 5 to 50 wt % buffering agent, from 40 to 95 wt % particles, and from 0 to 10 wt % linear polymer. Reformulation can be performed with an aqueous buffered diluent solution like that of Example 1.

1. A method of forming radiopaque crosslinked hydrogel particles comprising (a) mixing a reactive multi-arm polymer comprising a plurality of cyclic imide ester groups, a reactive multifunctional compound comprising a plurality of amino groups, and a radiocontrast agent under conditions in which a pH environment surrounding the reactive multi-arm polymer, the reactive multifunctional compound and the radiocontrast agent increases from an acidic pH to a basic pH, thereby forming a radiopaque hydrogel and (b) subjecting the hydrogel to a particle size reduction process, thereby forming the radiopaque crosslinked hydrogel particles.

2. The method of claim 1, wherein a first solution that is buffered at a first pH ranging from 3.5 to 6.0 and contains the reactive multi-arm polymer, the reactive multifunctional compound, and the radiocontrast agent is mixed with a second solution that is buffered at a second pH ranging from 8.0 to 12.0.

3. The method of claim 1, wherein a first solution that is buffered at a first pH ranging from 3.5 to 6.0 and contains the reactive multi-arm polymer and the reactive multifunctional compound is mixed with a second solution that is buffered at a second pH ranging from 8.0 to 12.0 and contains the radiocontrast agent.

4. The method of claim 1, wherein the mixing takes place in a planetary centrifugal mixer.

5. The method of claim 1, wherein the particle size reduction process comprises processing the hydrogel in a homogenizer.

6. The method of claim 1, further comprising filtering and/or sieving the radiopaque crosslinked hydrogel particles after being subjected to the particle size reduction process.

7. The method of claim 1, further comprising suspending the radiopaque crosslinked hydrogel particles in a carrier fluid to form a hydrogel particle suspension.

8. The method of claim 7, wherein the carrier fluid has a pH ranging from 3.5 to 6.0.

9. The method of claim 7, wherein the carrier fluid comprises a linear polymer.

10. The method of claim 9, wherein the linear polymer is selected from a polyalkylene oxide linear polymer, a polyoxazoline linear polymer, a polypyrrolidone linear polymer, a polyacrylamide linear polymer, and a polyhydroxyethylmethacrylate linear polymer and/or the linear polymer has number average molecular weight ranging from 5 to 20 kDa.

11. The method of claim 7, wherein the hydrogel particle suspension is contained in a preloaded syringe.

12. The method of claim 11, wherein the method further comprises packaging the preloaded syringe, a needle, and a luer connector in one or more sterile trays.

13. The method of claim 1, wherein the radiocontrast agent comprises an iodinated organic compound.

14. The method of claim 1, wherein the multi-arm polymer comprises three or more polymer arms linked to a core region, each of the polymer arms comprising a hydrophilic polymer segment and cyclic imide ester end group.

15. The method of claim 14, wherein each of the polymer arms comprises a hydrolysable ester group disposed between the hydrophilic polymer segment and the cyclic imide ester group and/or wherein the hydrophilic polymer segment is selected from polyalkylene oxide segments, polyester segments, polyoxazoline segments, polydioxanone segments, and polypeptide segments.

16. The method of claim 14, wherein the core region comprises a polyol residue.

17. The method of claim 1, wherein the reactive multifunctional compound is selected from a poly(amino acid) and a multi-arm amino-terminated PEG.

18. The method of claim 17, wherein the reactive multifunctional compound is trilysine.

19. Radiopaque crosslinked hydrogel particles, the particle formed by a process that comprises (a) mixing a reactive multi-arm polymer comprising a plurality of cyclic imide ester groups, a reactive multifunctional compound comprising a plurality of amino groups, and a radiocontrast agent under conditions in which a pH environment surrounding the reactive multi-arm polymer, the reactive multifunctional compound and the radiocontrast agent increases from an acidic pH to a basic pH, thereby forming a radiopaque hydrogel and (b) subjecting the hydrogel to a particle size reduction process, thereby forming the radiopaque crosslinked hydrogel particles.

20. A suspension of radiopaque crosslinked hydrogel particles, the suspension formed by a process that comprises (a) mixing a reactive multi-arm polymer comprising a plurality of cyclic imide ester groups, a reactive multifunctional compound comprising a plurality of amino groups, and a radiocontrast agent under conditions in which a pH environment surrounding the reactive multi-arm polymer, the reactive multifunctional compound and the radiocontrast agent increases from an acidic pH to a basic pH, thereby forming a radiopaque hydrogel, (b) subjecting the hydrogel to a particle size reduction process, thereby forming the radiopaque crosslinked hydrogel particles, and (c) suspending the radiopaque crosslinked hydrogel particles in a carrier fluid that comprises a linear polymer to form a hydrogel particle suspension.
